(19) United States
(12) Patent Application Publication
(10) Pub. No.: US 2025/0364076 A1
(43) Pub. Date: Nov. 27, 2025

(54) System For Patient Disease Monitoring and Method Thereof

(71) Applicant: CARDIAI TECHNILOGIES, Calgary (CA)

(72) Inventors: Anmol Singh Kapoor, Calgary (CA); Raja B. Singh, Calgary (CA); Sidharth Singh Bhinder, Calgary (CA)

(21) Appl. No.: 19/213,330

(22) Filed: May 20, 2025

Related U.S. Application Data
(60) Provisional application No. 63/650,209, filed on May 21, 2024.

(51) Int. Cl. G16B 20/20; G16B 40/30; G16B 50/30; G16H 15/00; G16H 50/30

(52) U.S. Cl. CPC G16B 20/20; G16B 40/30; G16B 50/30; G16H 15/00; G16H 50/30

(57) ABSTRACT

The present disclosure relates to a computer-implemented system (100) for patient disease monitoring using reference single nucleotide polymorphisms (SNPs) cluster identifications (rsIDs), comprising a data acquisition module (102) configured to collect raw data and convert into comma separated values (CSV) files. The system (100) also comprising a dedicated database (104) contains a list of reference single nucleotide polymorphisms (SNPs) cluster identifications (rsIDs). The system (100) also comprising a backend processing assembly (106) further comprising a data pre-processing module (108) configured to identify a plurality of disease-specific comma separated values (CSV) files. The backend processing assembly (106) further comprising a comparison module (110) and an intelligent analytic module (112). The system (100) also comprising an output interface (114) configured to provide comprehensive records of the reference single nucleotide polymorphisms (SNPs) cluster identifications (rsIDs) shared between the disease-specific datasets and the reference list.

### FIELD OF THE INVENTION

Embodiments of the present invention relate to field of genomics and specifically relates to a system for patient disease monitoring using genomics.

### DESCRIPTION OF THE RELATED ART

Recent developments in the field of genomics have revolutionized the understanding of disease susceptibility, enabling researchers and clinicians to uncover genetic variants associated with a wide range of health conditions. One of the foundational elements in this endeavor is the identification and analysis of single nucleotide polymorphisms (SNPs), which are commonly represented by Reference SNP cluster IDs (rsIDs). These genetic markers play a critical role in identifying inherited traits, assessing disease risk, and guiding personalized medical interventions.

A growing body of research has led to the development of large-scale, disease-specific genomic datasets, often curated in structured formats. These datasets catalog rsIDs known or suspected to be associated with specific diseases, such as diabetes, cancer, cardiovascular disorders, and neurological conditions. However, despite the availability of such data, there remains a significant gap in tools that enable efficient, automated comparison of individual genetic profiles against multiple disease-specific datasets.

Traditional approaches often rely on manual comparison, limited scope, or complex bioinformatics pipelines that are not accessible to non-specialists. Furthermore, there is no unified solution to determine whether a user's genetic profile contains rsIDs that are common across multiple disease types. While a variety of tools and platforms exist for analyzing genetic variants and associating them with disease risk, such solutions are riddled with numerous challenges and limitations including, focus on single disease, complex operational flow, lack of cross-referencing, lack of cross-platform integration, lack of user-friendly interface, and more.

Therefore, there is significant gap that requires improvements usability, scalability, and cross-disease genetic interpretation, for both research and precision medicine applications. Thus, the disclosed invention provides a solution for patient disease monitoring that provides insights into potential disease risks and comorbidities based on shared genetic variants.

### SUMMARY OF THE INVENTION

Embodiments of the present invention relate to a computer-implemented system for patient disease monitoring using reference single nucleotide polymorphisms (SNPs) cluster identifications (rsIDs). The system comprising a data acquisition module configured to collect raw data and convert into comma separated values (CSV) files. The comma separated values (CSV) files contains genetic data in the form of reference single nucleotide polymorphisms (SNPs) cluster identifications (rsIDs). The system also comprising a dedicated database operably connected to the data acquisition module and the dedicated database contains a list of reference single nucleotide polymorphisms (SNPs) cluster identifications (rsIDs). The system also comprising a backend processing assembly operably connected to the dedicated database and the data acquisition module and the backend processing assembly further comprising a data pre-processing module configured to identify a plurality of disease-specific comma separated values (CSV) files. The backend processing assembly further comprising a comparison module configured to compare the disease-specific comma separated values (CSV) files from the data pre-processing module with the list of reference single nucleotide polymorphisms (SNPs) cluster identifications (rsIDs) stored in the dedicated database. The backend processing assembly further comprising an intelligent analytic module configured to analyze the results of the comparison module and generate output files summarizing the results of the analysis. The system further comprising an output interface operably connected to the backend processing assembly, the output interface configured to provide comprehensive records of the reference single nucleotide polymorphisms (SNPs) cluster identifications (rsIDs) shared between the disease-specific datasets and the reference list. The output interface configured to organize the results on a per-disease basis. The output interface configured to facilitate a granular examination of genetic similarities across different disease types.

In accordance with an embodiment of the present invention, the output interface produces separate output files for each disease type.

In accordance with an embodiment of the present invention, the separate output files to facilitate analysis of shared genetic markers across diseases.

In accordance with an embodiment of the present invention, the comparison module identifies matching reference single nucleotide polymorphisms (SNPs) cluster identifications (rsIDs).

In accordance with an embodiment of the present invention, the intelligent analytic module leverages a plurality of data manipulation techniques.

In accordance with an embodiment of the present invention, the intelligent analytic module uncovers common genetic factors underlying various health conditions.

Another embodiment of the present invention relates to a computer-implemented method for patient disease monitoring using reference single nucleotide polymorphisms (SNPs) cluster identifications (rsIDs) and the method comprising receiving a plurality of comma separated values (CSV) files containing genetic data in the form of reference single nucleotide polymorphisms (SNPs) cluster identifications (rsIDs) via a data acquisition module. The method also comprising receiving and storing a list of reference single nucleotide polymorphisms (SNPs) cluster identifications (rsIDs) in a dedicated database. The method also comprising pre-processing the comma separated values (CSV) files via a data pre-processing module. The method also comprising comparing the each of the disease-specific comma separated values (CSV) files with the list of reference single nucleotide polymorphisms (SNPs) cluster identifications (rsIDs) via a comparison module. The method also comprising identifying the same reference single nucleotide polymorphisms (SNPs) cluster identifications (rsIDs). The method also comprising analyzing and generating output files summarizing the results via an intelligent analytic module.

In accordance with an embodiment of the present invention, the output files generated are comma separated values (CSV) files.

In accordance with an embodiment of the present invention, an empty comma separated values (CSV) file is generated, if no reference single nucleotide polymorphisms (SNPs) cluster identifications (rsIDs) matches are found.

In accordance with an embodiment of the present invention, the method also comprises providing the comprehensive records of the reference single nucleotide polymorphisms (SNPs) cluster identifications (rsIDs) shared between the disease specific datasets and the reference list via the output interface.

In accordance with an embodiment of the present invention, the method also comprises organizing results on the basis of the disease via the output interface.

In accordance with an embodiment of the present invention, the method also comprises facilitating a granular examination of genetic similarities across different disease types via the output interface.

In accordance with an embodiment of the present invention, the method also comprises storing or displaying via the output interface to facilitate analysis of genetic similarities across disease types.

In accordance with an embodiment of the present invention, the method also comprises generating insights into shared genetic variants.

### BRIEF DESCRIPTION OF THE DRAWINGS

So that the manner in which the above-recited features of the present invention is understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

The invention herein will be better understood from the following description with reference to the drawings, in which:

FIG. 1 illustrates a block diagram for a computer-implemented system 100 for patient disease monitoring using reference single nucleotide polymorphisms (SNPs) cluster identifications (rsIDs), in accordance with an embodiment of the present invention; and

FIG. 2 illustrates a flowchart for a computer-implemented method 200 for patient disease monitoring using reference single nucleotide polymorphisms (SNPs) cluster identifications (rsIDs), in accordance with an embodiment of the present invention.

It should be noted that the accompanying figure is intended to present illustrations of exemplary embodiments of the present disclosure. This figure is not intended to limit the scope of the present disclosure. It should also be noted that the accompanying figure is not necessarily drawn to scale.

### DETAILED DESCRIPTION OF THE INVENTION

In the following detailed description, numerous specific details are set forth in order to provide a thorough understanding of the embodiment of the invention as illustrative or exemplary embodiments of the invention, specific embodiments in which the invention may be practiced are described in sufficient detail to enable those skilled in the art to practice the disclosed embodiments. However, it will be obvious to a person skilled in the art that the embodiments of the invention may be practiced with or without these specific details. In other instances, well-known methods, procedures and components have not been described in detail so as not to unnecessarily obscure aspects of the embodiments of the invention.

The following detailed description is, therefore, not to be taken in a limiting sense, and the scope of the present invention is defined by the appended claims and equivalents thereof. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. References within the specification to “one embodiment,” “an embodiment,” “embodiments,” or “one or more embodiments” are intended to indicate that a particular feature, structure, or characteristic described in connection with the embodiment is included in at least one embodiment of the present invention.

Although the terms first, second, etc. may be used herein to describe various elements, these elements should not be limited by these terms. These terms are generally only used to distinguish one element from another and do not denote any order, ranking, quantity, or importance, but rather are used to distinguish one element from another. Further, the terms “a” and “a” herein do not denote a limitation of quantity, but rather denote the presence of at least one of the referenced items.

The conditional language used herein, such as, among others, “can,” “may,” “might,” “may,” “e.g.,” and the like, unless specifically stated otherwise, or otherwise understood within the context as used, is generally intended to convey that certain embodiments include, while other embodiments do not include, certain features, elements and/or steps.

Disjunctive language such as the phrase “at least one of X, Y, Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

### The Following Brief Definition of Terms Shall Apply Throughout the Present Invention

The terms “determining”, “measuring”, “evaluating”, “assessing,” “assaying,” and “analyzing” can be used interchangeably herein to refer to any form of measurement, and include determining if an element is present or not. (e.g., detection). These terms can include both quantitative and/or qualitative determinations. Assessing may be relative or absolute.

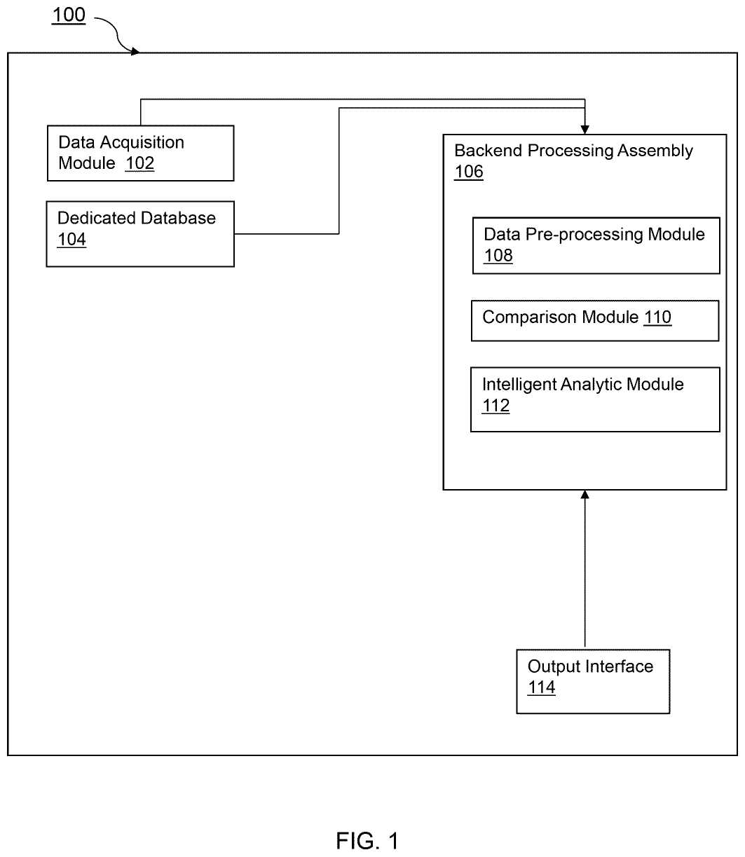

FIG. 1 illustrates a block diagram for a computer-implemented system 100 for patient disease monitoring using reference single nucleotide polymorphisms (SNPs) cluster identifications (rsIDs), in accordance with an embodiment of the present invention.

The system 100 may also comprising a data acquisition module 102, a dedicated database 104, a backend processing assembly 106, a data pre-processing module 108, a comparison module 110, an intelligent analytic module 112, and an output interface 114.

The data acquisition module 102 may be configured to collect raw data and convert into comma separated values (CSV) files. The comma separated values (CSV) files may contain genetic data in the form of reference single nucleotide polymorphisms (SNPs) cluster identifications (rsIDs).

In an embodiment of the present disclosure, the data acquisition module 102 may be collect raw genetic data from one or more sources, including but not limited to genotyping arrays, sequencing platforms, or third-party APIs. Upon collection, the data acquisition module 102 may preprocess the data to ensure compatibility with downstream processing modules. In a preferred embodiment, the data acquisition module 102 may convert the collected raw data into standardized comma-separated values (CSV) files. Each CSV entry may contain one or more fields such as rsID, chromosomal position, genotype, and so.

In some embodiments, the data acquisition module 102 may convert the collected raw data into some other standardized file format. The file generated may have interoperability with various bioinformatics tools, machine learning models, and storage or retrieval subsystems. In some embodiments, the data acquisition module 102 may be deployed in a standalone manner or integrated within the backend processing assembly 106.

The dedicated database 104 may be operably connected to the data acquisition module 102 and the dedicated database 104 contains a list of reference single nucleotide polymorphisms (SNPs) cluster identifications (rsIDs).

In an embodiment of the present disclosure, the dedicated database 104 may, either directly or via a communication network, connect to the data acquisition module 102. The dedicated database 104 may be configured to store a predefined list of reference single nucleotide polymorphism (SNP) cluster identifications (rsIDs) uploaded by a user, where rsIDs may serve as unique identifiers for genetic loci used in genomic analysis, annotation, or downstream interpretation.

In some embodiments, the dedicated database 104 may be implemented as a relational database, document store, or in-memory key-value store, depending on performance and scalability requirements. In some embodiments, the dedicated database 104 may contain metadata associated with each rsID, such as chromosomal location, known alleles, population frequency data, clinical relevance annotations, or references to any external genomic databases.

The backend processing assembly 106 may be operably connected to the dedicated database 104 and the data acquisition module 102 and the backend processing assembly 106 further comprising a data pre-processing module 108 configured to identify a plurality of disease-specific comma separated values (CSV) files.

In an embodiment of the present disclosure, the backend processing assembly 106 may be a microcontroller or a remote cloud-based server, capable of processing and handling large amount of data. In an embodiment of the present disclosure, the backend processing assembly 106 may manage computational tasks, and perform analytical operations on genetic data collected by the system 100.

In an embodiment of the present disclosure, the data pre-processing module 108 may identify and extract features from the plurality of disease-specific comma-separated values (CSV) files. In an embodiment of the present disclosure, the data pre-processing module 108 may perform filtering of the data based on reference SNP IDs (rsIDs) stored in the dedicated database 104, and segregating relevant data corresponding to specific diseases or genetic conditions.

In some embodiments, the data pre-processing module 108 may utilize rule-based logic, pattern-matching algorithms, or machine learning classifiers to map raw genotype records to disease-specific categories.

The comparison module 110 may be configured to compare the disease-specific comma separated values (CSV) files from the data pre-processing module with the list of reference single nucleotide polymorphisms (SNPs) cluster identifications (rsIDs) stored in the dedicated database.

The comparison module 110 may identify matching reference single nucleotide polymorphisms (SNPs) cluster identifications (rsIDs).

In an embodiment of the present disclosure, the comparison module 110 may perform a record-by-record analysis to determine the presence, absence, or variation of specific rsIDs within each disease-specific CSV file. The comparison module 110 may evaluate whether the rsIDs in the CSV files match known reference entries in the dedicated database 104 and may flag discrepancies, novel variants, or mutations of interest. In some embodiments, the comparison process may include genotype verification, allele frequency analysis, annotation enrichment, and mapping to known disease-related SNPs. In some embodiments, the comparison module 110 may assign confidence scores or classification labels based on the degree of match or deviation from reference data. In some embodiments, the comparison module 110 may log all matching and mismatching rsIDs for auditability, traceability, and future review. In some embodiments, the comparison module 110 may be implemented using high-performance computing techniques, such as parallelized search algorithms or in-memory databases, to handle large volumes of genomic data.

The intelligent analytic module 112 may be configured to analyze the results of the comparison module 110 and generate output files summarizing the results of the analysis.

The intelligent analytic module 112 may leverage a plurality of data manipulation techniques.

The intelligent analytic module 112 may uncover common genetic factors underlying various health conditions.

In an embodiment of the present disclosure, the intelligent analytic module 112 may apply one or more analytic algorithms, decision models, or statistical methods to interpret the outcomes of the comparison between disease-specific comma-separated values (CSV) files and the reference single nucleotide polymorphism (SNP) cluster identifications (rsIDs) stored in the dedicated database 104. The intelligent analytic module 112 may be configured to generate one or more output files summarizing the results of the analysis. In some embodiments, the intelligent analytic module 112 may employ artificial intelligence (AI) or machine learning (ML) techniques-such as clustering, classification, or predictive modelling, to provide higher-level insights, such as the likelihood of disease predisposition, comorbidity risks, and more.

In some embodiments, the intelligent analytic module 112 may generate diagnostic indicators, support risk assessments, or feed into downstream analytics or machine learning models.

The output interface 114 may be operably connected to the backend processing assembly, the output interface configured to provide comprehensive records of the reference single nucleotide polymorphisms (SNPs) cluster identifications (rsIDs) shared between the disease-specific datasets and the reference list. The output interface 114 may be operably connected to organize the results on a per-disease basis. The output interface 114 may be operably connected to facilitate a granular examination of genetic similarities across different disease types.

The output interface 114 may produce separate output files for each disease type.

The separate output files may facilitate analysis of shared genetic markers across diseases.

In an embodiment of the present disclosure, the output interface 114 may be any electronic device such as, but not limited to, mobile, laptop, personal computer, and more. In some embodiments, the output provided by the output interface 114 may include, but are not limited to, lists of matched and unmatched rsIDs, statistical metrics such as variant frequency, z-scores, or p-values, inferred disease risk scores or clinical interpretations; and visual summaries such as tables, charts, or genomic heat maps. In some embodiments, the output provided 112 may be formatted for compatibility with electronic health record (EHR) systems, third-party decision support platforms, or downstream reporting modules. The output may be stored locally or transmitted securely to authorized endpoints for further review, action, or integration.

In an embodiment of the present disclosure, the system 100 may leverage pandas library for data manipulation ensures robust handling of CSV files, enabling rapid analysis even with large datasets. The system's 100 design may prioritize efficiency and flexibility, allowing for seamless comparison of genetic data across diverse disease types.

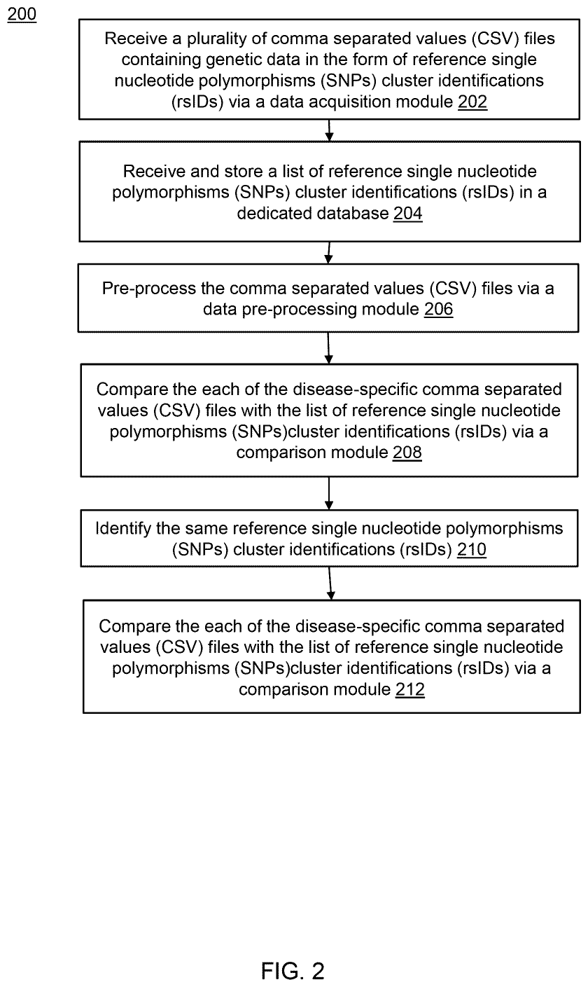

FIG. 2 illustrates a flowchart for a computer-implemented method 200 for patient disease monitoring using reference single nucleotide polymorphisms (SNPs) cluster identifications (rsIDs), in accordance with an embodiment of the present invention.

The method 200 may be comprising the following steps.

At 202, receiving a plurality of comma separated values (CSV) files containing genetic data in the form of reference single nucleotide polymorphisms (SNPs) cluster identifications (rsIDs) via a data acquisition module.

At 204, receiving and storing a list of reference single nucleotide polymorphisms (SNPs) cluster identifications (rsIDs) in a dedicated database.

At 206, pre-processing the comma separated values (CSV) files via a data pre-processing module.

At 208, comparing the each of the disease-specific comma separated values (CSV) files with the list of reference single nucleotide polymorphisms (SNPs) cluster identifications (rsIDs) via a comparison module.

At 210, identifying the same reference single nucleotide polymorphisms (SNPs) cluster identifications (rsIDs).

At 212, analyzing and generating output files summarizing the results via an intelligent analytic module.

The output files may be generated may be comma separated values (CSV) files.

An empty comma separated values (CSV) file may be generated, if no reference single nucleotide polymorphisms (SNPs) cluster identifications (rsIDs) matches are found.

The method 200 may also comprise providing the comprehensive records of the reference single nucleotide polymorphisms (SNPs) cluster identifications (rsIDs) shared between the disease specific datasets and the reference list via the output interface 114.

The method 200 may also comprise organizing results on the basis of the disease via the output interface 114.

The method 200 may also comprise facilitating a granular examination of genetic similarities across different disease types via the output interface 114.

The method 200 may also comprise storing or displaying via the output interface 114 to facilitate analysis of genetic similarities across disease types.

The method 200 may also comprise generating insights into shared genetic variants.

In an embodiment of the present disclosure, the method 200 may accept a user-provided list of rsIDs and efficiently compare across multiple disease-specific genetic datasets. In an embodiment of the present disclosure, the method 200 may automate the identification of overlapping rsIDs across diseases without requiring advanced bioinformatics expertise. In an embodiment of the present disclosure, the method 200 may provide structured output that highlights shared genetic markers in a format amenable to further interpretation or integration with clinical systems.

The disclosed invention streamlines the comparison of user-provided genetic data in terms of the CSV with disease-specific datasets, offering novel insights into potential disease risks and comorbidities based on shared genetic variants. The disclosed invention may be beneficial in revealing shared pathogenic mechanisms, informing comorbidity risks, or highlighting pleiotropic genetic variants, which are critical in precision medicine and clinical diagnostics. The disclosed invention may enable clinicians, researchers, and patients to better understand cross-disease genetic associations from individual genotypic data.

In a case that no conflict occurs, the embodiments in the present disclosure and the features in the embodiments may be mutually combined. The foregoing descriptions are merely specific implementations of the present disclosure, but are not intended to limit the protection scope of the present disclosure. Any variation or replacement readily figured out by a person skilled in the art within the technical scope disclosed in the present disclosure shall fall within the protection scope of the present disclosure. Therefore, the protection scope of the present disclosure shall be subject to the protection scope of the claims.

The foregoing descriptions of specific embodiments of the present technology have been presented for purposes of illustration and description. They are not intended to be exhaustive or to limit the present technology to the precise forms disclosed, and obviously many modifications and variations are possible in light of the above teaching. The embodiments were chosen and described in order to best explain the principles of the present technology and its practical application, to thereby enable others skilled in the art to best utilize the present technology and various embodiments with various modifications as are suited to the particular use contemplated. It is understood that various omissions and substitutions of equivalents are contemplated as circumstance may suggest or render expedient, but such are intended to cover the application or implementation without departing from the spirit or scope of the claims of the present technology.

What is claimed for:

1. A computer-implemented system for patient disease monitoring using reference single nucleotide polymorphisms (SNPs) cluster identifications (rsIDs), the system comprising:
a data acquisition module configured to collect raw data and convert into comma separated values (CSV) files,
wherein the comma separated values (CSV) files contains genetic data in the form of reference single nucleotide polymorphisms (SNPs) cluster identifications (rsIDs);
a dedicated database operably connected to the data acquisition module, the dedicated database contains a list of reference single nucleotide polymorphisms (SNPs) cluster identifications (rsIDs);
a backend processing assembly operably connected to the dedicated database and the data acquisition module, the backend processing assembly further comprising:
a data pre-processing module configured to identify a plurality of disease-specific comma separated values (CSV) files;
a comparison module configured to compare the disease-specific comma separated values (CSV) files from the data pre-processing module with the list of reference single nucleotide polymorphisms (SNPs) cluster identifications (rsIDs) stored in the dedicated database; and
an intelligent analytic module configured to analyze the results of the comparison module and generate output files summarizing the results of the analysis; and
an output interface operably connected to the backend processing assembly, the output interface configured to:
provide comprehensive records of the reference single nucleotide polymorphisms (SNPs) cluster identifications (rsIDs) shared between the disease-specific datasets and the reference list;
organize the results on a per-disease basis; and
facilitate a granular examination of genetic similarities across different disease types.

2. The system of claim 1, wherein the output interface produces separate output files for each disease type.

3. The system of claim 2, wherein the separate output files to facilitate analysis of shared genetic markers across diseases.

4. The system of claim 1, wherein the comparison module identifies matching reference single nucleotide polymorphisms (SNPs) cluster identifications (rsIDs).

5. The system of claim 1, wherein the intelligent analytic module leverages a plurality of data manipulation techniques.

6. The system of claim 1, wherein the intelligent analytic module uncovers common genetic factors underlying various health conditions.

7. A computer-implemented method for patient disease monitoring using reference single nucleotide polymorphisms (SNPs) cluster identifications (rsIDs), the method comprising:
receiving a plurality of comma separated values (CSV) files containing genetic data in the form of reference single nucleotide polymorphisms (SNPs) cluster identifications (rsIDs) via a data acquisition module;
receiving and storing a list of reference single nucleotide polymorphisms (SNPs) cluster identifications (rsIDs) in a dedicated database;
pre-processing the comma separated values (CSV) files via a data pre-processing module;
comparing the each of the disease-specific comma separated values (CSV) files with the list of reference single nucleotide polymorphisms (SNPs) cluster identifications (rsIDs) via a comparison module;
identifying the same reference single nucleotide polymorphisms (SNPs) cluster identifications (rsIDs); and
analyzing and generating output files summarizing the results via an intelligent analytic module.

8. The method of claim 7, wherein the output files generated are comma separated values (CSV) files.

9. The method of claim 8, wherein an empty comma separated values (CSV) file is generated, if no reference single nucleotide polymorphisms (SNPs) cluster identifications (rsIDs) matches are found.

10. The method of claim 8, wherein the method also comprises providing the comprehensive records of the reference single nucleotide polymorphisms (SNPs) cluster identifications (rsIDs) shared between the disease specific datasets and the reference list via the output interface.

11. The method of claim 10, wherein the method also comprises organizing results on the basis of the disease via the output interface.

12. The method of claim 10, wherein the method also comprises facilitating a granular examination of genetic similarities across different disease types via the output interface.

13. The method of claim 10, wherein the method also comprises storing or displaying via the output interface to facilitate analysis of genetic similarities across disease types.

14. The method of claim 7, wherein the method also comprises generating insights into shared genetic variants.
